(19) United States
(12) Patent Application Publication
(10) Pub. No.: US 2009/0246880 A1
(43) Pub. Date: Oct. 1, 2009

(54) Method for preventing degradation of diacetylpolyamine

(76) Inventor: Masaru Hamaoki, Choshi-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/382,965

(22) Filed: Mar. 27, 2009

(30) Foreign Application Priority Data
Mar. 28, 2008 (JP) 2008-085098

(51) Int. Cl. G01N 33/50; C07C 225/06

(52) U.S. Cl. 436/106; 564/502

(57) ABSTRACT

The present invention provides a method for preventing degradation of diacetylpolyamine in a solution containing diacetylpolyamine, the method including adding a proteinase inhibitor and/or a peptidase inhibitor to the solution, or lowering the pH of the solution. Employment of the method of the present invention realizes inhibition of degradation of diacetylpolyamine in a solution containing diacetylpolyamine, and determination of more accurate assay data on diacetylpolyamine. The method of the present invention is widely applicable to, for example, a sample containing proteinase or peptidase (e.g., urine or cell lysate), or an assay reaction mixture which may unintentionally contain proteinase or peptidase. Therefore, the method is very useful for assay of diacetylpolyamine.

### BACKGROUND OF THE INVENTION

1. Field of the Invention

The present invention relates to a method for preventing degradation of diacetylpolyamine in a solution containing diacetylpolyamine; to a solution in which degradation of diacetylpolyamine has been inhibited through the method; and to a method for assaying diacetylpolyamine by use of the solution.

2. Background Art

Diacetylpolyamine has been known as a marker which is correlated with the malignancy of cancer (J. Biochem. (Tokyo) 2006, 139: 315-322). For example, diacetylspermine, which is a type of diacetylpolyamine, has been used as a diagnostic marker for determining the degree of malignancy of a tumor (WO 2004/081569).

Such a diacetylpolyamine is a physicochemically stable substance, and a specific oxidase which generally metabolizes an acetylated polyamine (Int. J. Biochem. 1981, 13: 287-292) does not exhibit high activity in a common living-organism-derived sample. Therefore, hitherto, no particular attention has been paid to degradation of diacetylpolyamine in a solution containing diacetylpolyamine.

However, as a result of extensive studies on stability of diacetylpolyamine, the present inventor has found that diacetylpolyamine contained in a sample derived from a living organism is degraded not by a hitherto considered specific oxidase, but by a commonly present proteinase or peptidase, and thus assay of the diacetylpolyamine may fail to yield accurate data.

### SUMMARY OF THE INVENTION

In order to develop a method for preventing degradation of diacetylpolyamine, the present inventor has conducted extensive studies, and as a result has found that when a proteinase inhibitor and/or a peptidase inhibitor is added to a solution containing diacetylpolyamine, or when the pH of a solution containing diacetylpolyamine is adjusted to 6.5 or lower, degradation of diacetylpolyamine contained in the solution can be reduced. The present invention has been accomplished on the basis of this finding. Accordingly, the present invention provides the following.

(1) A method for preventing degradation of diacetylpolyamine in a solution containing diacetylpolyamine, the method comprising adding a proteinase inhibitor and/or a peptidase inhibitor to the solution.

(2) A method for preventing degradation of diacetylpolyamine in a solution containing diacetylpolyamine, the method comprising adjusting the pH of the solution to 6.5 or lower.

(3) A method for preventing degradation of diacetylpolyamine in a solution containing diacetylpolyamine, the method comprising adding a proteinase inhibitor and/or a peptidase inhibitor to the solution, and adjusting the pH of the solution to 6.5 or lower.

(4) A solution containing diacetylpolyamine whose degradation has been inhibited through a method as recited in any of (1) to (3) above.

(5) A solution as described in (4) above, which is a living-organism-derived sample that has been prepared for assay of diacetylpolyamine.

(6) A method for assaying diacetylpolyamine, comprising using, as a sample to be assayed, a solution as recited in (4) above.

Employment of the method of the present invention realizes inhibition of degradation of diacetylpolyamine in a solution containing diacetylpolyamine, and determination of more accurate assay data on diacetylpolyamine. The method of the present invention is widely applicable to, for example, a sample which contains or is highly likely to contain proteinase or peptidase (e.g., urine or cell lysate), or an assay reaction mixture which may unintentionally contain proteinase or peptidase. Therefore, the method is very useful for assay of diacetylpolyamine.

### DETAILED DESCRIPTION OF PREFERRED EMBODIMENTS

As used herein, the term “diacetylpolyamine” refers to diacetylspermine and/or diacetylspermidine. Specific examples include N¹,N¹²-diacetylspermine and N¹,N⁸-diacetylspermidine.

No particular limitation is imposed on the “solution containing diacetylpolyamine,” so long as the solution contains diacetylpolyamine. Specific examples of the solution include living-organism-derived samples such as urine, serum, plasma, and cell lysate; and a reaction mixture used for assay of diacetylpolyamine.

As used herein, the expression “prevention of degradation” refers to the case where, as described in the Examples hereinbelow, the percent degradation of diacetylpolyamine is 15% or less when a solution containing diacetylpolyamine is incubated at 37° C. for two hours. The expression “inhibition of degradation” has the same meaning as the expression “prevention of degradation.” From the viewpoint of assay of diacetylpolyamine, the lower the percent degradation of diacetylpolyamine, the better. However, a percent diacetylpolyamine degradation of 15% or less does not greatly affect assay data (in particular, data obtained through immunoassay).

As described above, in the method of the present invention for preventing degradation of diacetylpolyamine in a solution containing diacetylpolyamine, a proteinase inhibitor and/or a peptidase inhibitor is added to the solution, and/or the pH of the solution is adjusted to 6.5 or lower.

No particular limitation is imposed on the proteinase inhibitor or peptidase inhibitor employed in the present invention, so long as it inhibits proteinase or peptidase activity. Specific examples of the inhibitor include protease inhibitor cocktails P2714 and P8340 (products of Sigma), Bestatin, and carboxypeptidase inhibitors.

The method for addition of any of the aforementioned inhibitors, the amount of the inhibitor employed, etc. may be determined on the basis of the instruction attached to the inhibitor, or already reported methods in relation to the inhibitor.

No particular limitation is imposed on the method for lowering the pH of a solution containing diacetylpolyamine, so long as the method does not affect assay of diacetylpolyamine. Specifically, a buffer having a target pH (e.g., tris-HCl buffer or phosphate buffer) may be added to a solution containing diacetylpolyamine, to thereby adjust the pH of the solution to 6.5 or lower (preferably 6 or lower).

The thus-prepared diacetylpolyamine-containing solution in which degradation of diacetylpolyamine has been inhibited is suitable as a sample for determination of diacetylpolyamine concentration. When such a sample is employed, more accurate assay data on diacetylpolyamine can be obtained.

A variety of methods for assaying diacetylpolyamine have already been reported. Among these methods, immunoassay is particularly simple and preferred. Specifically, immunoassay of diacetylpolyamine may be carried out according to known publications (see, for example, Japanese Patent Application Laid-Open (kokai) No. 2000-074917; WO 2004-81569; J. Cancer Res. Clin. Oncol., 123 (1997), 539-545; or J. Biochem. (Tokyo), 132 (2002), 783-788).

### EXAMPLES

The present invention will next be described in detail by way of examples, which should not be construed as limiting the invention thereto. The percent degradation of diacetylspermine (%) in a sample was calculated on the basis of the amount of diacetylspermine as determined through ELISA.

### Example 1

### Inhibition of Degradation of Diacetylspermine through Addition of Proteinase Inhibitor or Peptidase Inhibitor to a Solution Containing Diacetylspermine

Mouse hemocytes were washed and then disrupted through freezing and thawing, and the disrupted hemocytes were dissolved in saline so that the disrupted hemocyte concentration of the solution was 5%. Diacetylspermine was added to the thus-prepared mouse hemocyte lysate so that the diacetylspermine concentration of the mixture was 80 μM, and the pH of the mixture was adjusted to 7.4 with 25 mM tris-HCl buffer. As a proteinase (or peptidase) inhibitor, protease inhibitor cocktail P2714 (product of Sigma) was dissolved in 2 mL of water and added to the reaction mixture so that the inhibitor concentration of the mixture was 0.2 vol %. Similarly, P83401 (product of Sigma) was added to the reaction mixture so that the inhibitor concentration of the mixture was 1 vol %. In either case, the reaction mixture was incubated at 37° C. for two hours.

Before incubation at 37° C. and after completion of incubation with or without addition of the inhibitor, the amount of diacetylspermine contained in each sample was determined through ELISA. Subsequently, the ratio of the amount of diacetylspermine degraded through incubation to the initial amount of diacetylspermine was calculated.

Table 1 shows comparison in percent degradation of diacetylspermine between the case where no proteinase (or peptidase) inhibitor was added and the case where each of the aforementioned proteinase (or peptidase) inhibitors was added. As is clear from Table 1, degradation of diacetylspermine is prevented through addition of the proteinase (or peptidase) inhibitor.

TABLE 1

| | Inhibitor | | |
|---|---|---|---|
| | None | P2714 | P8340 |
| Percent degradation (%) | 32.2 | 3.1 | 7.2 |

### Example 2

### Inhibition of Degradation of Diacetylspermine through Adjustment of the pH of a Solution Containing Diacetylspermine

A mouse hemocyte lysate was prepared in a manner similar to that described above in Example 1, and diacetylspermine was added to the lysate so that the diacetylspermine concentration of the mixture was 40 μM. Subsequently, the pH of the mixture was adjusted with 50 mM phosphate buffer to 5.5, 6.0, 6.5, 7.0, 7.5, or 8.0. Thereafter, the reaction mixture was incubated at 37° C. for two hours.

Before incubation at 37° C. and after completion of incubation, the amount of diacetylspermine contained in each sample was determined through ELISA in a manner similar to the case of Example 1. Subsequently, the ratio of the amount of diacetylspermine degraded through incubation to the initial amount of diacetylspermine was calculated.

Table 2 shows the percent degradation of diacetylspermine relative to the pH of the reaction mixture. As is clear from Table 2, degradation of diacetylspermine is prevented by adjusting the pH of the reaction mixture to 6.5 or lower (preferably 6 or lower).

TABLE 2

| | pH of reaction mixture | | | | | |
|---|---|---|---|---|---|---|
| | 5.5 | 6.0 | 6.5 | 7.0 | 7.5 | 8.0 |
| Percent degradation (%) | 0.0 | 5.2 | 11.1 | 25.9 | 62.6 | 78.3 |

What is claimed is:

1. A method for preventing degradation of diacetylpolyamine in a solution containing diacetylpolyamine, the method comprising adding a proteinase inhibitor and/or a peptidase inhibitor to the solution.

2. A method for preventing degradation of diacetylpolyamine in a solution containing diacetylpolyamine, the method comprising adjusting the pH of the solution to 6.5 or lower.

3. A method for preventing degradation of diacetylpolyamine in a solution containing diacetylpolyamine, the method comprising adding a proteinase inhibitor and/or a peptidase inhibitor to the solution, and adjusting the pH of the solution to 6.5 or lower.

4. A solution containing diacetylpolyamine whose degradation has been inhibited through a method as recited in claim 1.

5. A solution as recited in claim 4, which is a living-organism-derived sample that has been prepared for assay of diacetylpolyamine.

6. A method for assaying diacetylpolyamine, comprising using, as a sample to be assayed, a solution as recited in claim 4.
